(19) United States
(12) Patent Application Publication
(10) Pub. No.: US 2003/0013950 A1
(43) Pub. Date: Jan. 16, 2003

(54) Dual isotope studies in nuclear medicine imaging

(76) Inventors: David Rollo, Saratoga, CA (US); Horace Hines, San Jose, CA (US); David E. Coles, San Francisco, CA (US)

Correspondence Address: CORPORATE PATENT COUNSEL, PHILIPS ELECTRONICS NORTH AMERICA CORP., 580 WHITE PLAINS ROAD, TARRYTOWN, NY, 10591, US

(21) Appl. No.: 09/894,276

(22) Filed: Jun. 27, 2001

(51) Int. Cl. A61B 5/05

(52) U.S. Cl. 600/407

(57) ABSTRACT

A gamma camera system is described in which counts are scatter corrected using multiple windows located in the vicinity of a photopeak. Pixel data is thereby scatter corrected prior to image formation. The system is useful-in nuclear medicine studies using dual isotopes such as stress and lung perfusion studies, wherein count data of the multiple isotopes are acquired simultaneously.

This invention relates to nuclear medicine (gamma camera) imaging systems and, in particular, to the conduct of dual isotope studies.

Dual isotope studies have been conducted in nuclear medicine to elicit different kinds of clinical information during the same study. In a dual isotope study, two radionuclides are administered to the patient prior to the imaging session, with each radionuclide being specific to a different type of anatomy or physiological function. The energy peaks for both emissions are detected during the imaging acquisition process and separately binned to form an image for each radionuclide. The clinician can thereby make a diagnosis based upon the integration of the information obtained from the results produced by the different radionuclides.

A problem which arises during such dual isotope studies is erroneous event counts due to Compton scattering. The energy of one radionuclide can scatter and produce background noise in the vicinity of a lower energy peak of another radionuclide. This background noise will be incorrectly recorded as event counts at the lower energy peak, resulting in inaccurately reconstructed images. In the past, attempts at correcting for this scattering have focused on image processing techniques. It would be desirable however to correct for this scattering during the acquisition process, so that corrected images can be produced without the need for further processing and correction.

In accordance with the principles of the present invention, a gamma camera system acquires event data from multiple radionuclides during the same study. The events are acquired in multiple energy windows. The event counts in the multiple windows are combined to produce corrected pixel data, which is then used to produce an image. Thus, scatter correction is performed during the acquisition process. The present invention finds useful application in multiple radionuclide studies of the heart and lungs.

In the drawings:

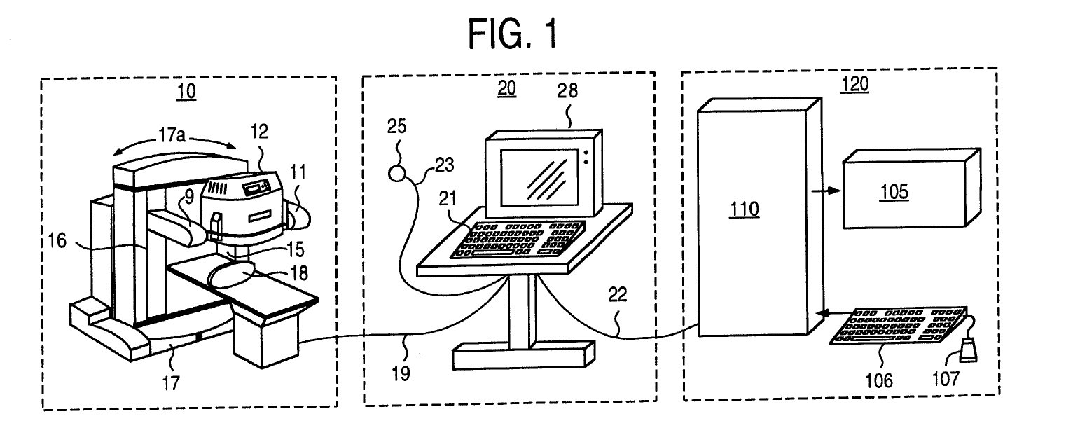

FIG. 1 illustrates the major components of a gamma camera system;

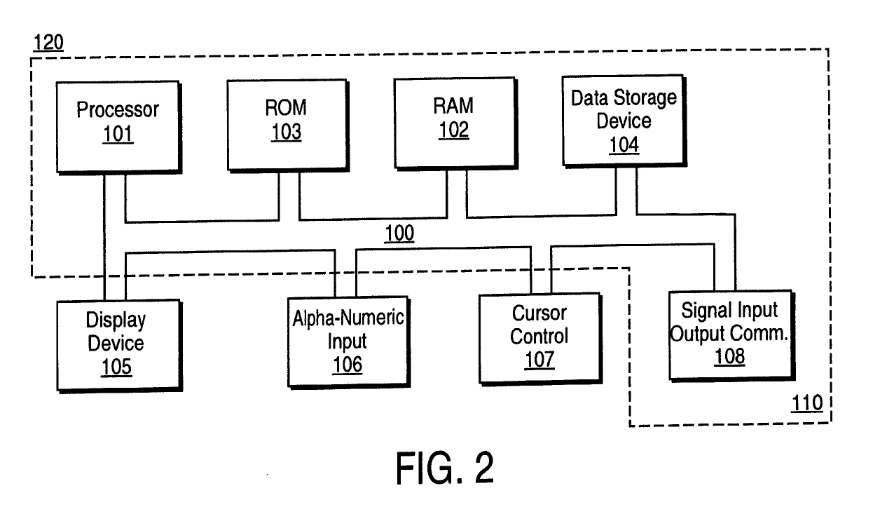

FIG. 2 illustrates in block diagram form the post data acquisition processing and display system of the gamma camera of FIG. 1;

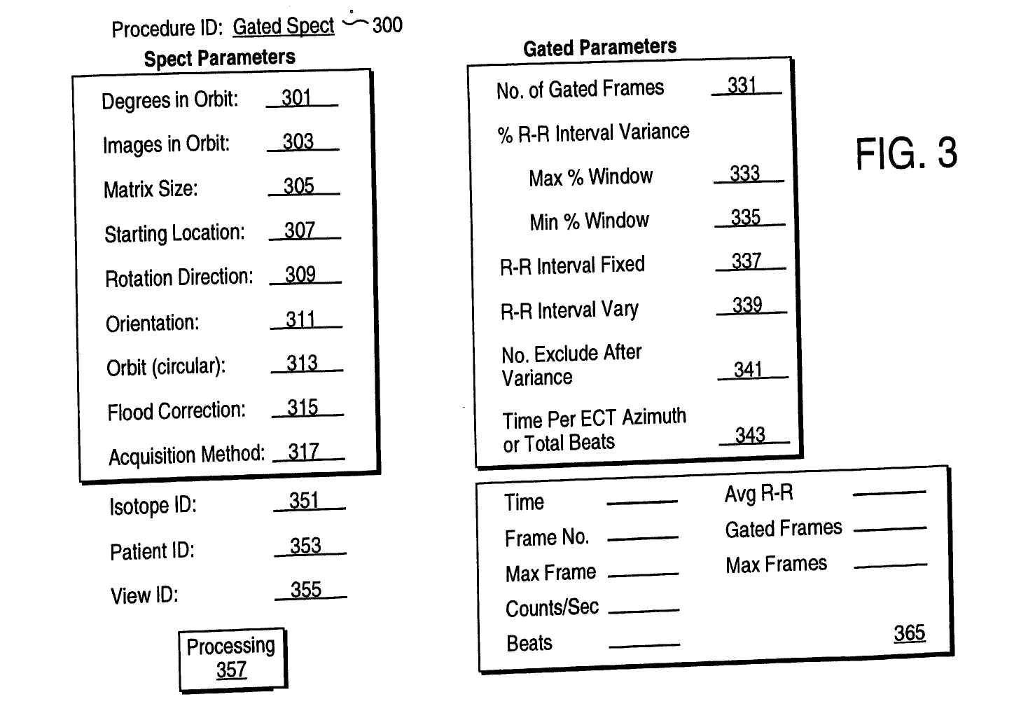

FIG. 3 illustrates some of the parameters which may be used in a gated SPECT study;

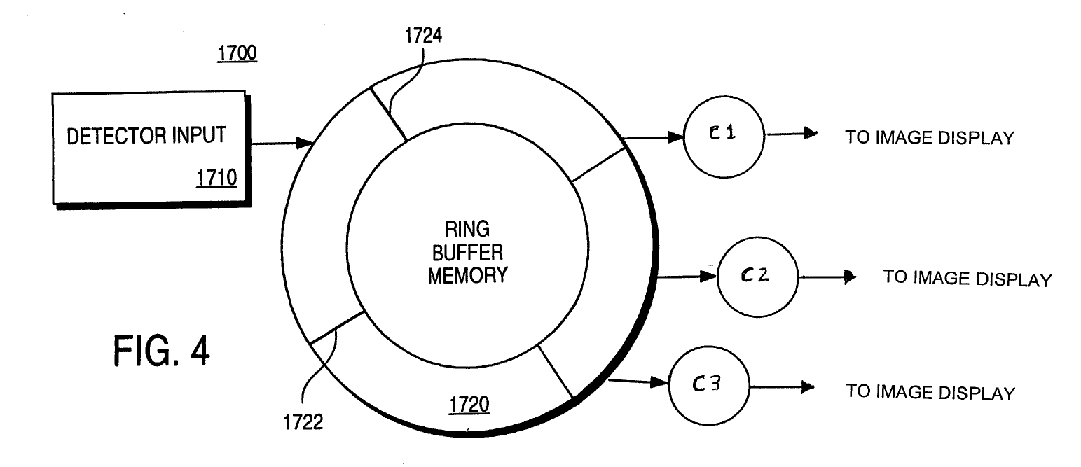

FIG. 4 illustrates in block diagram form a network of the gamma camera which simultaneously processes different data sets from the same imaging procedure in accordance with the principles of the present invention;

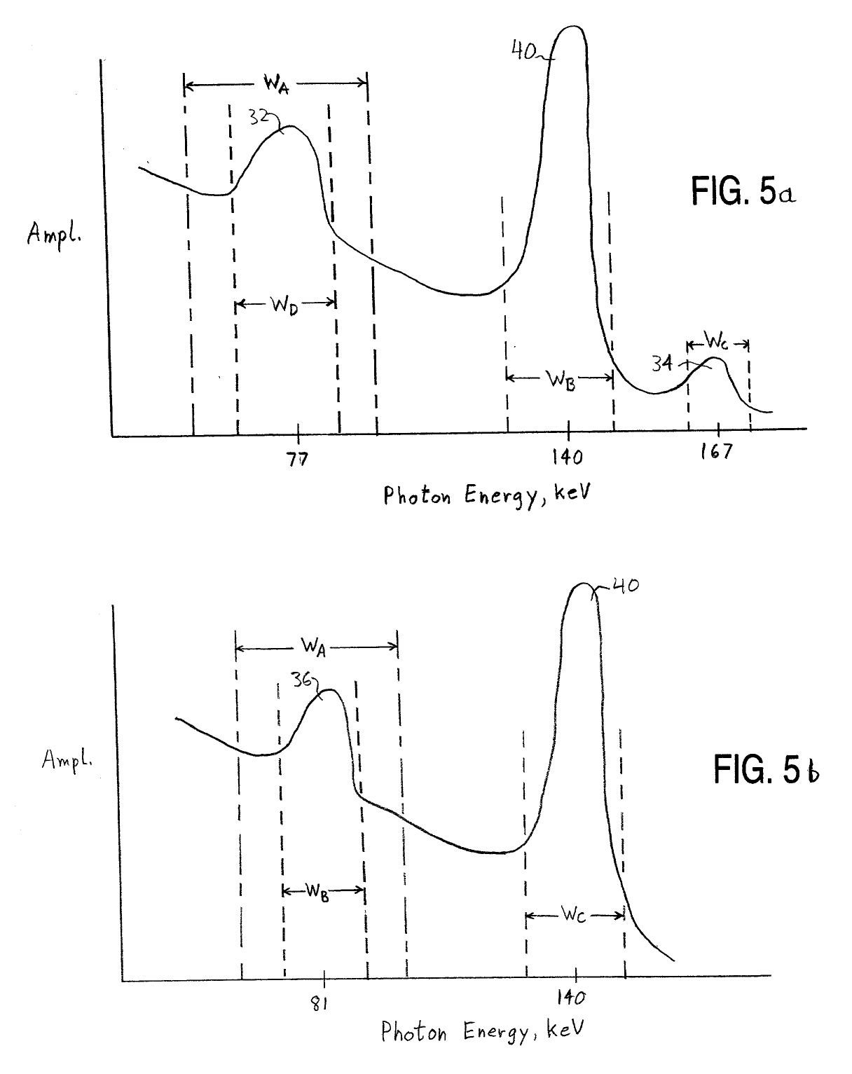

FIGS. 5a and 5b illustrate energy peaks and windows for heart and lung studies which use multiple radionuclides;

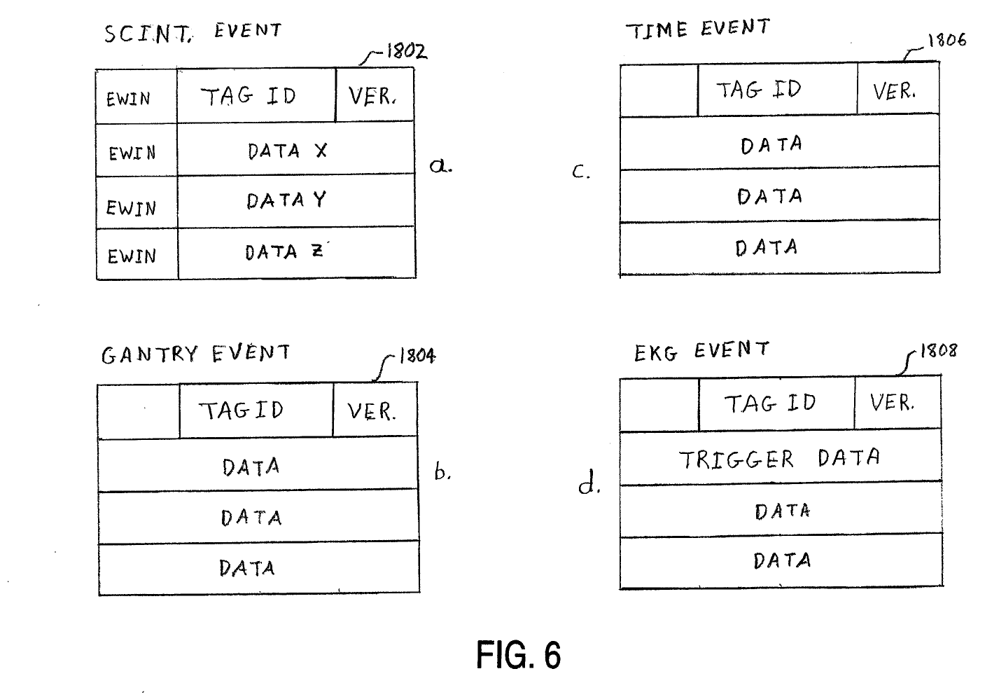

FIGS. 6a-6d illustrate the format of the data used in a constructed embodiment of the present invention; and

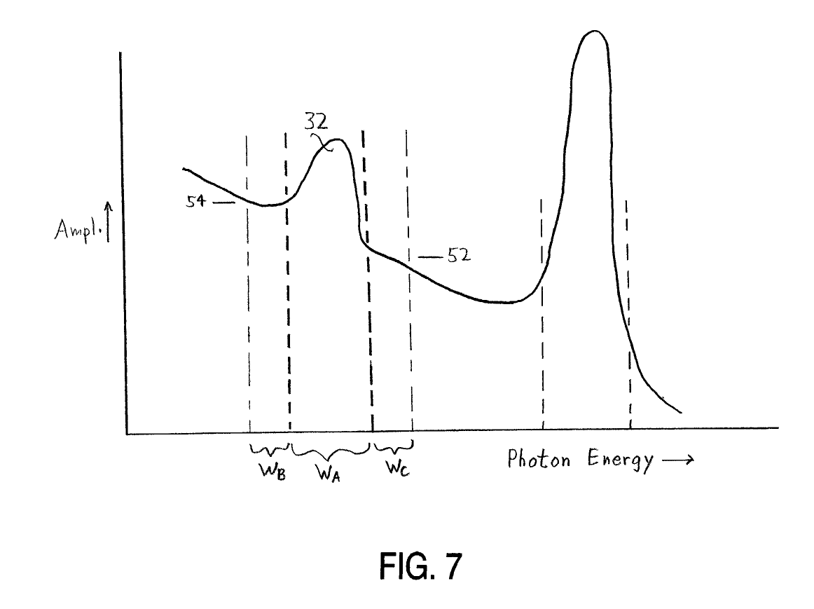

FIG. 7 illustrates another scattering correction technique using multiple energy windows.

FIG. 1 illustrates the major components of a nuclear camera image acquisition, processing and display system. The present invention includes either a single head (single detector) camera 10 as shown in the drawing or a dual head (dual detector) camera as shown in U.S. Pat. No. 5,760,402 (Hug. et al.) or U.S. Pat. No. 6,150,662 (Hug et al.). These camera systems are SPECT cameras ideal for cardiac, abdominal, and whole body studies and are capable of implementing gated SPECT imaging techniques. In the illustration of FIG. 1, two arms 11 and 9 mounted on vertical tracks 16 and 15 form a gantry structure that can move the detector head 12 in various projection angles to accomplish the required 180 and 360 degree movements of the detector 12 used in gated SPECT and other studies. Pivot structure 17 allows the camera detector 12 and gantry structure to pivot clockwise or counterclockwise. The camera system 10 includes a detector head 12 comprising a number of well known radiation detection components of the Anger camera type including an array of photomultiplier tubes, a collimator, a scintillating crystal and a digital pixel output. The camera system 10, in a well known fashion, images the patient to provide digital image data which is binned according to particular discrete angles of rotation in which the detector 12 traverses about the patient. Binning can also occur according to particular phases of the cardiac cycle (R-R interval, defined below). For each angle of rotation, several phases of the cardiac cycle may be interrogated. Particular (x, y) coordinate positions within the imaging detector of the camera system are called pixel locations and the number of scintillations detected by each pixel location is represented by a count value for that pixel. Each pixel contains a count value representing the number of radiation emissions detected at that location of the detector 12. The resulting digital image data from the camera system 10 is binned according to the particular discrete angle of rotation in which the detector was situated when the image data was acquired. Also binned is the gated segment (phase) within the R-R interval in which the data was acquired in gated SPECT studies. The pixel matrix of (x, y) locations is referred to herein as a histogram of scintillations at these coordinate locations. It is understood that a histogram represents a raw image. For example, a typical detector 12 may have a resolution of (64×64) pixels or (128×128) pixels available for imaging and is capable of imaging at a maximum resolution of approximately (1000×1000) pixels.

The camera system 10 is coupled to a data acquisition computer system 20, which in a particular constructed embodiment is implemented using a general purpose computer system having high speed communications ports for input and output coupled to a two-way data transmission line 19 coupling the camera system 10 to the computer system 20. The computer system 20 communicates data acquisition parameters (also called data acquisition protocols) selected by a user to the camera system 10 to initiate a particular type of study by the camera system 10. The imaging data from the camera system 10 is then transferred over line 19 to the communications device of the system 20 and this raw gated SPECT image data is then forwarded to a post acquisition processing computer system 120. The data acquisition system 20 also comprises a keyboard entry device 21 for user interface to allow selection and modification of predefined data acquisition parameters which control the imaging processes of the camera system 10. Also coupled to the data acquisition system 20 is a standard color display monitor 28 for display of parameter information and relevant information regarding the particular gated SPECT study underway such as imaging status communicated from the camera system 10 during an imaging session.

For a gated SPECT study a cardiac electrode and signal amplification unit 25 is also coupled to the data acquisition computer system 20, and the cardiac signal goes directly to the acquisition computer 10. This unit 25 is specially adapted to couple with a patient's chest near the heart to receive the heartbeat electrical signal. The unit 25 is composed of well known heartbeat detection and amplification (EKG) components and any of several well known devices can be utilized within the scope of the present invention. In order to perform gated SPECT analysis on the heart, the heartbeat pulse or electrical wave must be studied for each patient, as each patient's cardio rhythm is different. The Aft heartbeat waveform is examined to determine the points within the cycle where the well-known R wave is encountered. The time interval between successive R waves is measured to determine the R-R interval. These points and timing intervals between these points will be used to gate the imaging process of the camera system 10 during the cardiac cycle. The preferred embodiment of the present invention automatically, under control of the system 20, collects five sample heartbeat waves once the detector 25 is located on the subject patient in order to determine the average R-R period. This information is fed to the computer system 20 and then sent to the camera system 10. However such information could also be detected and determined directly by the computer system 10 once conditioned to do so by the acquisition computer system 20 under user control. For a particular projection angle, the system 10 directs the acquired imaging counts to the first segment bin, and upon each successive time interval the image data is directed to a new gated bin. When the R wave is detected once more, the first bin receives the image data again and the process continues through each other segment and associated bin until a new projection angle is encountered. The electrode 25 also is used by the camera system 10 in order to detect the start of a cardiac cycle and gate the camera imaging system appropriately depending on the number of selected segments of the R-R interval used for collection.

As discussed above, the data acquisition portion of the imaging system is composed of camera system 10 and computer system 20. Referring still to FIG. 1, the image data is sent from the camera system 10 over line 19 to acquisition system 20 and then over line 22 to the post acquisition processing system 120. This system 120 is responsible for processing, displaying and quantifying certain data acquired by system 10 and system 20.

The post acquisition processing system 120 accepts the raw gated SPECT image data generated by the camera system 10 and, using user configurable procedures, reconstructs (produces tomographic images) the data to provide a reconstructed volume and from the volume generates specialized planar or volumetric images for diagnosis, including generating and displaying the functional images as described above. In cardiac imaging the generated images or frames represent different slices of the reconstructed heart volume at variable thicknesses in a short axis dimension, a vertical dimension and a horizontal dimension (all three are user configurable) for a number of gated time segments. Therefore, complete three dimensional information can be displayed by display 105 in a two dimensional manner in a variety of formats and orientations.

The computer of the post acquisition processing system 120 in a constructed embodiment illustrated in FIG. 2 is a SPARC system available from Sun Microsystems of California, however any number of similar computer systems having the requisite processing power and display capabilities will suffice within the scope of the present invention. Generally, the system 120 comprises a bus 100 for communicating information, a central processor 101 coupled with the bus for processing information (such as image data and acquired counts) and command instructions, a random access memory 102 coupled with the bus 100 for storing information and instructions for the central processor 101, a read only memory 103 coupled with the bus 100 for storing static information and command instructions for the processor 101, a data storage device 104 such as a magnetic disk or optical disk drive coupled with the bus 100 for storing information (such as both raw gated SPECT and reconstructed data sets) and command instructions, and a display device 105 coupled to the bus 100 for displaying information to the computer user. There is also an alphanumeric input device 106 including alphanumeric and function keys coupled to the bus 100 for communicating information and command selections to the central processor 101, a cursor control device 107 coupled to the bus for communicating user input information and command selections to the central processor 101 based on hand movement, and an input and output device 108 coupled to the bus 100 for communicating information to and from the computer system 120. The input and output device 108 includes, as an input device, a high speed communication port configured to receive image data acquired by the nuclear camera system 10 and fed over line 22.

The display device 105 utilized with the system of the present invention may be a liquid crystal device, cathode ray tube, or other display device suitable for creating graphic images and alphanumeric characters recognizable to the user. The display unit 105 of the preferred embodiment of the present invention is a high resolution color monitor. The cursor control device 107 allows the computer user to dynamically signal the two dimensional movement of a visible symbol or cursor (pointer) on a display screen of the display device 105. Many implementations of the cursor control device are known in the art including a trackball, mouse, joystick or special keys on the alphanumeric input device 105 capable of signaling movement of a given direction or manner of displacement. It will be appreciated that the cursor control device 107 also may be directed and/or activated via input from the keyboard using special keys and key sequence commands, or from a touchscreen display device. In the discussions regarding cursor movement and/or activation within the preferred embodiment, it is to be assumed that the input cursor directing device may consist of any of those described above and is not limited to the mouse cursor device. It will be appreciated that the computer chassis 110 may include the following components of the image processor system: the processor 101, ROM 103, RAM 102, the data storage device 104, and the signal input and output communication device 108 and optionally a hard copy printing device.

The data acquisition system 20 allows a user via keyboard control to select and/or create a predefined set of parameters (or protocols) for direction of a gated SPECT imaging session or other selected study by the camera system 10. FIG. 3 illustrates a parameter interface screen and configurable parameters of a nuclear camera system for data acquisition that are selected and displayed on a screen by the user via keyboard 21. FIG. 3 illustrates some of the parameters that are configurable by the data acquisition system 20. It is appreciated that once set, the configurable parameters can be saved and referenced in a computer file for subsequent recall. The stored parameters or protocol file can then be recalled and utilized for a particular study, thus eliminating the need to reenter the parameters for similar or identical studies. The name of the parameter file shown in FIG. 3 is “GATED SPECT” and is indicated at 300. It is appreciated that the computer system 20, once instructed by the user, will relay the parameters set by the user to the camera system 10 in order to initialize and begin a particular study. The initiation is done by selection of processing command 357. A user interface of this type is thus versatile while at the same time providing a high degree of automation of the execution of selected study protocols.

In accordance with the principles of the present invention, the gamma camera system of FIGS. 1-3 is capable of producing images from several radionuclides during the same study by use of the data network shown in FIG. 4. The network includes a ring buffer 1720 into which gamma camera data is entered at a high data rate. The data in the illustrated ring buffer 1720 may have a specified start point 1722 and an end point 1724 that may adjust around the ring buffer as data is received and processed. The gamma camera data is entered into the ring buffer by a Producer, one of which is shown at 1700. A Producer is a camera subsystem or data path which enters data into the ring buffer 1720. The Producer illustrated in the drawing is a data stream 1710 from a detector or camera head, which inputs detector data into the ring buffer. Other Producers may provide data from other sources such as stored data sources, for example. Some of the types of data words which are provided by a detector are described in FIG. 6 below.

Accessing the data which traverses the ring buffer 1720 are one or more Consumers. Three Consumers are shown in FIG. 4, and are labeled C1, C2, and C3. A Consumer is a data processor or path or other entity which makes use of some or all of the data in the ring buffer 1720. In the illustrated embodiment each Consumer is an entity conditioned to look for specific characteristics of event data and to read data from the ring buffer selected for a particular type of study. The studies in the following examples are all associated with types of images and hence the Consumers shown in this example read and process selected data into images, which can then be forwarded to an image display. Each Consumer C1, C2 and C3 examines the data in the ring buffer as it passes by its input, and independently reads those data words which are needed for the imaging process being supported by that Consumer. The Consumers operate both independently and simultaneously, and each can support one or more imaging processes.

Examples of the types of event data which may be provided by a detector are shown in FIG. 6. In this example each event word is 64 bits long. The words in this drawing are shown in four lines of sixteen bits each. FIG. 6a illustrates a scintillation event word 1802 with four energy window bytes EWIN of four bits each. The setting of one of these bits denotes one of sixteen energy windows in which the particular scintillation event was acquired. Typically a detector will only produce data for energy windows chosen by the camera operator. The TAG ID and TAG VERSION (VER.) bytes identify the data word as a scintillation event word. The TAG bytes provide information such as the detector number which produced the event to enable acquisitions from systems with multiple detectors. Data X and Data Y provide the x and y coordinate locations on the detector at which the event was sensed. The Data Z byte provides the energy number of the detected event.

FIG. 6b shows a format for a gantry event word 1804. Gantry event words provide information as to the current position and velocity of the gantry and hence the locations of the detectors. Gantry event data originates with sensors, controllers, and other devices associated with the gantry or from control programs for the gantry. The illustrated gantry event word 1804 has TAG ID and VER. bytes which identify the word as a gantry event word. The TAG bytes provide information as to the type of information contained in the gantry event word. The last three lines contain the data pertinent to the gantry event.

FIG. 6c gives an example of a time event word 1806. The acquisition system provides these words as time markers so that the other events of the camera can be oriented in time. Time events occur in regular intervals such as once every millisecond. The TAG bytes of the time event word denote the word as a time event word. The rest of the time event word comprises data giving the time information.

FIG. 6d illustrates an EKG event word 1808, which will be produced when a cardiac electrode unit 25 is used for a gated study. The TAG bytes identify the word as an EKG event word. A TRIGGER DATA byte provides information as to the trigger event, and the other data bytes of the EKG event word provide other information pertinent to the EKG event.

Other event words may also be present in the data stream provided by the detectors and entered into the ring buffer 1720. For example Start and Stop event words may be used to indicate the start of an image acquisition session and the conclusion of an image acquisition session.

The nuclear camera system described above can be advantageously used to conduct studies with multiple radionuclides as illustrated below. Stress studies, by which ischemia can be identified, is one application where multiple radionuclides may be used. In a stress study a patient exercises on a treadmill or stationary bicycle or is injected with a cardiovascular stimulant such as dobutamine until the heart rate reaches approximately 85% of the target stress rate. The patient is then injected with a radiopharmaceutical agent which localizes in the myocardium such as technetium-labeled sestamibi. The patient continues to exercise for another minute or so while the agent makes several passes through the heart and the myocardium takes up the agent. The agent may be imaged within the following 8-15 minutes to acquire images of the infused heart under stress conditions.

The patient returns for another imaging session after radiopharmaceutical agent has dissipated from the patient's body, which is generally about 24 hours after the first session. With the heart at a resting heart rate the patient is injected with the radiopharmacological agent again. The agent perfuses the myocardium when the heart is at rest and the patient is imaged once again. The clinician can then compare the stress and rest images. Cold areas in the stress images due to ischemia will be filled in the rest images, enabling the clinician to diagnose the ischemic condition.

In a preferred implementation of the present invention, the study is performed with two different radionuclides. The patient undergoes exercise or pharmacological stress until the 35% stress level is attained, and is injected with Tc-labeled sestamibi. A preferred radionuclide is technetium-99 m, and the Tc-labeied sestamibi, having a high extraction fraction in myocardial tissue, will once again perfuse the myocardium under stress. The patient exercises for another minute or so to enable the radiopharmacological agent to pass several times through the heart. The high affinity of sestamibi for the myocardial tissue causes the agent to persist in the tissue; after several hours, only about 1% of the agent will have redistributed. The patient is allowed to rest until a normal heart rate is attained. A thallium-201 (Tl) radionuclide is then injected and allowed to perfuse the myocardium for 10-15 minutes, at which time the patient is imaged.

The myocardial tissue now contains Tc which was trapped in the tissue by its biochemical carrier during stress. The tissue also contains Tl distributed in the myocardium during rest. The acquisition sequence now acquires scintillation events from these two radionuclides by looking for their different energy peaks. This is done by doing windowed acquisition about the energy peaks as illustrated in FIG. 5a. Tc has an energy peak 40 at 140 keV as illustrated in the drawing. The Tc is detected by locating a window W_{B} about the peak energy point and detecting scintillation events occurring within this energy window. Events in this window W_{B} are recorded in a scintillation event data word with the EWIN_{B} bit set to mark the word as an event from the energy window W_{B}.

The Tl has two energy peaks 32 and 34, one at 167 keV and another at 77 keV. To detect the thallium a window is located about each of these energy peaks, and events occurring in both windows W_{D} and W_{C} are aggregated to form the total number of counts from Tl. However the energy peaks are seen to be on an ever-increasing baseline of scatter noise as one proceeds from higher to lower energy levels. This is due to Compton scattering from the higher energy levels, which manifests itself as scatter events at lower energies. Since scattering occurs from higher to lower energy levels, the scatter background builds continually higher through the lower energy levels. For the counts to be accurate they should all be corrected for a constant baseline. That is, the number of counts need to be corrected for scatter.

In accordance with the principles of the present invention the counts for the Tl energy peak at 77 keV are scatter corrected by acquiring events in a second energy window W_{A} located about the 77 keV photopeak. The two measurements are then mathematically combined to produce a count total for the photopeak which is scatter corrected. The scatter corrected pixel data is then used to form an image. There are several ways in which the data from the two windows can be combined, depending upon the size of the windows, their degree of overlap, and the precision desired for the correction. One expression for the illustrated application is

P77=SumD−W_{D}*(SumA−SumD)/(W_{A}−W_{D})

where P77 is the sum of the counts in the photopeak at 77 keV corrected for scatter, SumA is the summation of the counts in the window A, SumD is the summation of the counts in the window D, W_{A} is the width of window A in energy channels and W_{D} is the width of window D in energy channels. The quotient on the right side of this expression scales the correction to account for the different widths of the energy windows W_{A} and W_{D}. The detector acquires two dimensional pixel data that is summed into projections. The corrections are preferably made for the counts in each pixel using the summed projection data.

The network of FIG. 4 sorts the event data from the above study in the following manner. The flow of data words into the ring buffer 1720 from the detector Producer 1710 will contain scintillation event data from all three energy levels (77 keV, 140 keV, and 167 keV), which is identified in Data Z of the scintillation event words. The four windows are identified by the setting of bits in the EWIN fields of the scintillation event words. As the event words traverse the ring buffer, the Consumers C1, C2, C3, etc. identify and read the data words for the respective images they support. For instance, four Consumers can read the data from the four separate windows, and the Consumers selecting windows A and D would combine their acquisition data to perform scatter correction, then combining this data with that from window C to obtain the total Tl counts prior to forwarding the data for image processing. Another possibility is for Consumer C1 to read the data from the 77 keV photopeak, Consumer C2, to read the data from the 140 keV photopeak, and Consumer C3 to read the data from the 167 keV photopeak. Consumers C1 and C3 combine their Tl data before image processing. A third possibility is for Consumer C1 to select all scintillation events for Tl (the 77 and 167 keV photopeaks) and for Consumer C2 to select all scintillation events for Tc (the 140 keV photopeak). An image for Tl would be produced from the data of Consumer C1 and an image for Tc would be produced from the data of Consumer C2.

Whereas FIG. 5a illustrates the spectra that is used for scatter correction using overlapping energy windows, FIG. 7 illustrates a scatter correction spectra example where energy windows which do not overlap are combined. By careful setting of the windows, scaling of the results is accomplished directly. In the FIG. 7 example an energy window W_{A} is set around the photopeak 32. This window W_{A} has a predetermined width in energy channels. Windows W_{B} and W_{C} are set on either side of window W_{A} with each having a width which is half that of window W_{A}. Additionally, when the background scatter increases approximately linearly as shown in the drawing, events in window W_{B} will exhibit a nominal energy level 54 and events in window W_{C} will exhibit a nominal energy level 52. The scatter baseline of the photopeak 32 is approximately halfway between these levels. Thus, the subtraction of the counts in windows W_{B} and W_{C} from the counts of window W_{A} will approximately cancel the scatter counts in the photopeak window W_{A} due to the relative scaling of the window sizes.

Another application where the present invention is particularly useful is a planar lung perfusion study. Such a study can have life-saving implications, as the diagnosis can be to identify a pulmonary embolus or blood clot in the lungs. An embolus is usually treated immediately with blood thinners, but these compounds can have their own harmful side effects such as inducing cerebral bleeding. Blockages similar to emboli can be present due to chronic obstructive lung disease, which manifests itself much like scar tissue. Hence it is desirable to quickly and positively identify the problem as an embolus and not chronic blockage, so that the blood thinners are not administered needlessly.

In accordance with the principles of the present invention, a lung perfusion study is performed with two radionuclides and a single imaging procedure. A carrier of macro-aggregated albumin is labeled with Tc and injected into the patient. This carrier becomes trapped in small capillaries in the lung, thereby trapping the Tc within the lungs on the basis of blood flow. Over time the albumin will metabolize and leave the system.

With the Tc in place in the capillaries, the patient inhales Xenon gas, preferably containing the radionuclide Xenon-133. The Xe will thus be distributed within the lungs on the basis of aeration rather than blood flow. Gamma camera imaging is then performed as the patient is breathing the Xe gas. Simultaneously acquired Tc and Xe images enable the clinician to identify the embolus, if present.

The photopeaks of the two radionuclides of this study are shown in FIG. 5b. Xe has a photopeak 36 at 81 keV, and Tc has a photopeak 40 at 140 keV. Scattering from the Tc will increase the background scatter at the Xe photopeak as the drawing illustrates. The Xe counts are corrected for scatter by the same windowing techniques and Consumer handling as described in FIG. 5a. Unlike FIG. 5a, each radionuclide in FIG. 5b has one photopeak, with the counts for each photopeak producing a separate image of Xe and Tl, respectively. The counts in windows W_{A} and W_{B} around the Xe photopeak 36 are combined to scatter correct the Xe acquisition data on a pixel by pixel basis, and the pixels are then forwarded to the image processor for display.

What is claimed is:

1. A nuclear camera system in which pixel data is scatter corrected prior to image processing comprising:
an acquisition subsystem which acts to acquire counts in the vicinity of a photopeak in multiple energy windows, including a scatter corrector which acts to correct for scatter in real time by mathematically combining the counts of the multiple energy windows; and
an image processor coupled to the scatter corrector which produces an image from scatter corrected count data.

2. The nuclear camera system of claim 1, wherein the acquisition subsystem acts to simultaneously acquire counts from multiple radionuclides producing emissions at different energy levels.

3. The nuclear camera system of claim 2, wherein the radionuclide producing emissions at the higher energy level produces background scatter at the photopeak at the lower energy level.

4. The nuclear camera system of claim 3, wherein the radionuclides are used in a stress study.

5. The nuclear camera system of claim 4, wherein the radionuclides are Tc and Tl.

6. The nuclear camera system of claim 3, wherein the radionuclides are used in a lung perfusion study.

7. The nuclear camera system of claim 6, wherein the radionuclides are Tc and Xe.

8. The nuclear camera system of claim 1, wherein the act of mathematically combining is an additive process.

9. The nuclear camera system of claim 1, wherein the act of mathematically combining is a subtractive process.

10. The nuclear camera system of claim 1, wherein the scatter corrector acts to correct for scatter on a pixel by pixel basis.

11. The nuclear camera system of claim 1, wherein the multiple energy windows are overlapping.

12. The nuclear camera system of claim 1, wherein the multiple energy windows occupy adjacent energy channels.

13. A method for performing a nuclear medicine lung perfusion study comprising:
applying a first carrier labeled with a first radionuclide to the blood flow system which becomes distributed in capillaries of the lungs;
applying a second carrier labeled with a second radionuclide to the lungs by inhalation; and
imaging both radionuclides simultaneously with a gamma camera.

14. The method of claim 13, wherein the first carrier is macro-aggregated albumin.

15. The method of claim 14, wherein the first radionuclide is Tc.

16. The method of claim 13, wherein the second carrier is a gas.

17. The method of claim 16, wherein the second radionuclide is Xe.

18. The method of claim 13, wherein imaging is performed while the second labeled carrier is being applied.

19. The method of claim 13, wherein imaging comprises producing a first nuclear image of a radionuclide distributed in a lung on the basis of blood flow; and
producing a second nuclear image of a radionuclide distributed in a lung on the basis of aeration.
